(19) United States
(12) Patent Application Publication
(10) Pub. No.: US 2008/0154217 A1
(43) Pub. Date: Jun. 26, 2008

(54) Puncture Tips And Catheter Tubes

(75) Inventors: Jean-Luc Carrez, Ecouen (FR); Valery Dalle, Gouvieux (FR)

Correspondence Address: LEVINE & MANDELBAUM, 444 MADISON AVENUE, 35TH FLOOR, NEW YORK, NY, 10022, US

(73) Assignee: VYGON, Ecouen (FR)

(21) Appl. No.: 11/815,347

(22) PCT Filed: Feb. 2, 2006
(86) PCT No.: PCT/FR2006/000231

§ 371 Date: Oct. 25, 2007

(30) Foreign Application Priority Data
Feb. 3, 2005 (FR) 0501072

(51) Int. Cl. A61M 5/32

(52) U.S. Cl. 604/272

(57) ABSTRACT

The invention relates to puncture tips which are used in the medical field. The puncture tip E comprises a hollow tube segment terminating in a cutting bevel which has a non-cutting state and which can return to said state by means of the shape memory effect when subjected to a prevalent condition in the site or under the effect of a stimulus controlled from the exterior, such as to be atraumatic. The invention is suitable for use in the medical field.

The invention concerns the needle-tips used in the medical field in order to penetrate the skin at a site, for the introduction of a catheter or a probe into the site for example.

Patent FR 2 809 625 proposed the constitution of a tapered needle-tip at the rounded distal end of a metal puncture needle for medical use, by means of a drop of an inert material deposited on this end by a dipping process and, after hardening, forming a sharp point that is designed to dissolve in bodily fluids, so that the channel of the needles open at this end after dissolving of the needle-tip, so as to allow the injection of a substance, and so that medical staff cannot prick themselves when removing the needle after use and during disposal.

This solution gives rise in to the following comments particular:
the formation of a sharp point by dipping can be insufficient to ensure correct puncture;
   the design of such a needle-tip is incompatible with a needle-tip that is manufactured with the tube;
dissolving of the needle-tip in the body can be quite undesirable, in particular in the case of a vein;
in the event of imperfect dissolving of the needle-tip, the tube can remain blocked;
dissolving of the needle-tip is difficult to check before withdrawal of the needle.

Publication JP 2000-354626 also proposed the creation of a needle for a puncture device with a cutting bevel in a shape-memory material, so that the channel section of the bevel is modified under the effect of body temperature.

The purpose of this present invention is to supply a needle-tip with a cutting bevel that is capable of deformation in the body until it becomes atraumatic, in controlled conditions, with no loss of material, without risk of occlusion of the tube fitted with the needle-tip, and suitable for manufacture with the tube.

Such a needle-tip is intended particularly to constitute the distal end of a catheter tube, in particular for the introduction of the catheter into a vein or an artery. Another aim of the invention is to supply a tube fitted with such a needle-tip and whose rigidity is able to vary in service, in a controlled manner between a rigidity that suffices so that the tube with its needle-tip constitutes a puncture needle, and a lower rigidity so that the tube has the flexibility of a catheter tube.

The puncture tip of the invention is composed of a section of hollow tube in a shape-memory material that, at its distal end, has a stable and rigid cutting bevel that is suitable to cut into the skin and penetrate into the site, characterised in that the cutting bevel remembers a state in which it is not sharp, and in that it is capable of changing state so as to return to this atraumatic state when it is subjected to a condition that is prevalent at the site or under the action of a stimulus controlled from the exterior.

The bevel can become atraumatic by means of a loss of rigidity and/or by adopting an atraumatic form.

It is preferable that the change of state of the bevel should be triggered by the temperature within the site or by a higher temperature created artificially.

This triggering method is not limiting, and other agents for triggering the change of configuration include, for example, the pH at the site, contact with the blood or with another liquid at the site, or electrical stimulation controlled from the exterior of the site.

Many solutions have already been proposed for the manufacture of a shape-memory tube for medical use, and there are a large number of publications on this subject. By way of some examples, one can mention the following publications in particular, which themselves make reference to other publications:
publication U.S. Pat. No. 5,674,242 describes polymer tubes for endoprosthetic devices composed of a shape-memory material formulated to include a state transition that is activated by a thermal stimulus;
publication U.S. Pat. No. 5,964,744 describes medical polymer compounds with shape memory for the manufacture of catheters for easy implantation in the urethra and designed to adopt a given configuration there;
publication WO 86/03980 describes several examples of shape-memory polymer compounds that soften at predetermined temperatures;
publication WO 02/085 188 describes a metal needle for medical use that has a distal end which retains the memory of a curved configuration, and that returns to this configuration when it is implanted, thus allowing it to bypass an obstacle;
publication WO 99/42 172 describes an implantable structure that includes a composite tube that is capable of deforming after implantation in the body. This tube includes segments in a heat-sensitive, shape-memory polymer material. The deformation is designed to allow the tube to adapt to difficult myocardial structures;
publication WO 95/08 296 describes a metal suturing needle that is intended for endoscopic operations, with the needle being straight at normal temperature and having the memory of a curved shape to which it returns at body temperature;
publication WO 96/11 721 describes a shape-memory catheter that can change from a rigid configuration to a flexible configuration;
   publication U.S. Pat. No. 6,059,815 describes a therapeutic actuator in polyurethane-based shape-memory material whose transformation is triggered by temperature;
   publication EP 0 422 693 describes catheters with shape memory that change configuration under the effect of temperature and of the processes employed to manufacture these catheters;
publication EP 0 931 559 describes an epidural catheter whose distal end retains the memory of a J-shape;
publication US 2002/01 42 119 describes several examples of materials with shape memory for use in manufacture;
publication FR 2 641 692 describes a plug in a polymer shape-memory material which is deformable under the effect of temperature in order to close off a breach in a medical application.

These publications only provide a general impression of particular uses of shape memory in the medical field, and those skilled in the art will be in possession of much more comprehensive general documentation concerning the materials and the processes that can be used for the implementation of shape memory.

By way of examples, one could mention former publications U.S. Pat. No. 4,053,548 and U.S. Pat. No. 4,193,899 which describe the means to impart heat-sensitive shape memory to particular elastomer materials.

A variety of materials have been recommended, such as alloys of nickel or copper (Ni−Ti, Cu−Al−Ni, Ni−Al, Mn−Cu, Fe−Ni, Cu—Zn−Al, Ni−Mn−Ga and Nitinoll™ in particular, polyvinyl chlorides, block copolymers allied to graft copolymers, sulfonated elastomer compounds, polynorbornenes, butadiene-styrene copolymers, polyurethanes, urethane polyether, polyamide polyethers, polycarbonates, derivatives of cellulose, and so on.

This present invention more particularly envisages the use of shape-memory plastic materials, composed of several polymers that are moulded into a first shape, namely that desired during use, and which is then stressed into a second shape before use. The latter is imposed by one of the polymers. On the triggering of a given phenomenon, the material imposing the second shape gives way, and the product returns to its first shape. The possible triggers can include temperature, liquids, UV light, etc.

To manufacture the needle-tip of the invention, with heat-sensitive shape memory, it is possible, amongst other things, to implement a process in which:
the needle-tip is moulded from a polymer material that has a selected phase transformation temperature, imparting to it the desired atraumatic shape;
the needle-tip is heated to a temperature that is greater than phase transformation temperature so as to soften it, and the softened needle tip is forced to assume the shape wanted for the puncture procedure;
   the needle tip is cooled to ambient temperature.

The needle-tip, thus prepared, is able to retain the shape in which it can act as a puncture tip, but in a vein or an artery returns to the atraumatic shape, provided that the phase transformation temperature exists within the site, and then retains its atraumatic shape when removed from the vein and the body.

It is preferable that the needle-tip should be created by moulding to the end of a tube to which it is attached.

The atraumatic shape of the bevel of the needle-tip can vary according to design and application. For example, it can be a rounding of the exit opening of the bevel (instead of being oblong and pointed in the cutting configuration), a balling of the bevel of the needle-tip (instead of being sharp in the cutting configuration), a curvature of the bevel of the needle-tip toward its opening, etc.

A description follows of various implementations of a tubular needle-tip for a needle that can be used in venous puncture and according to this present invention, with reference to the figures in the appended drawings in which:

FIG. 1 represents a needle-tip E whose cutting bevel is of the “Lancet Point” type, meaning a simple bevel created by milling to an angle, with this needle-tip being shown in profile (FIG. 1A) and face-on (FIG. 1B);

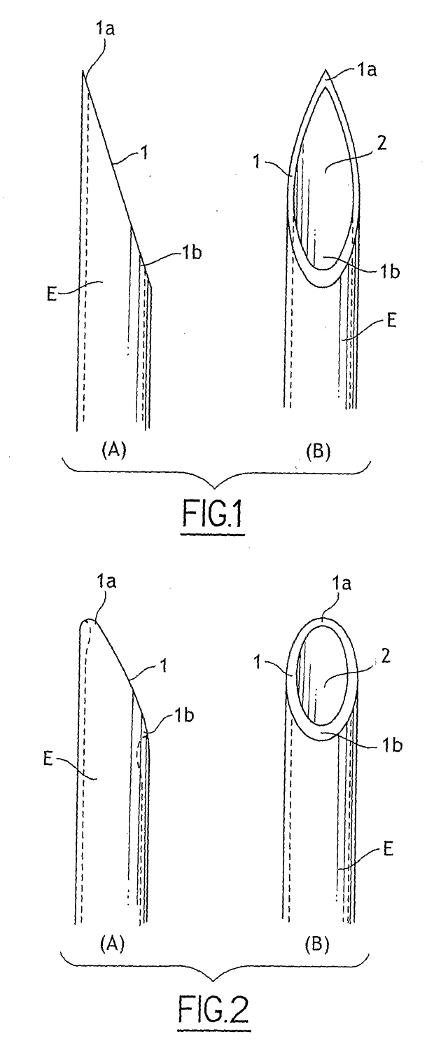

FIG. 2 represents the needle-tip of FIG. 1 after deformation, with this needle-tip being shown in profile (FIG. 2A) and face-on (FIG. 2B);

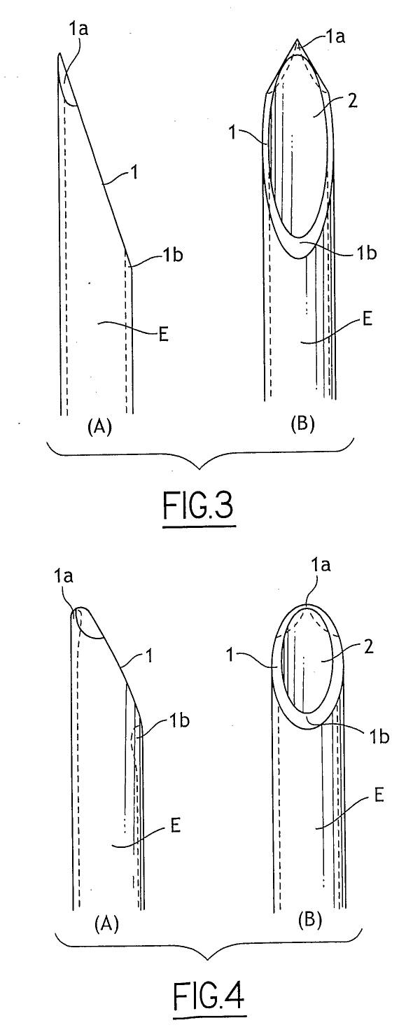

FIG. 3 represents a needle-tip (E) whose cutting bevel is of the “Back Cut” type, meaning a lancet-point bevel with two ground edges just behind the point, with this needle-tip being shown in profile (FIG. 3A) and face-on (FIG. 3B);

FIG. 4 represents the needle-tip of FIG. 3 after deformation, this needle-tip being shown in profile (FIG. 4-A) and face-on (FIG. 4-B);

FIGS. 5 to 8 represent in profile (figures A) and face-on (figures B), various cases of deformation of a cutting needle-tip E (Lancet Point or Back Cut), according to the invention;

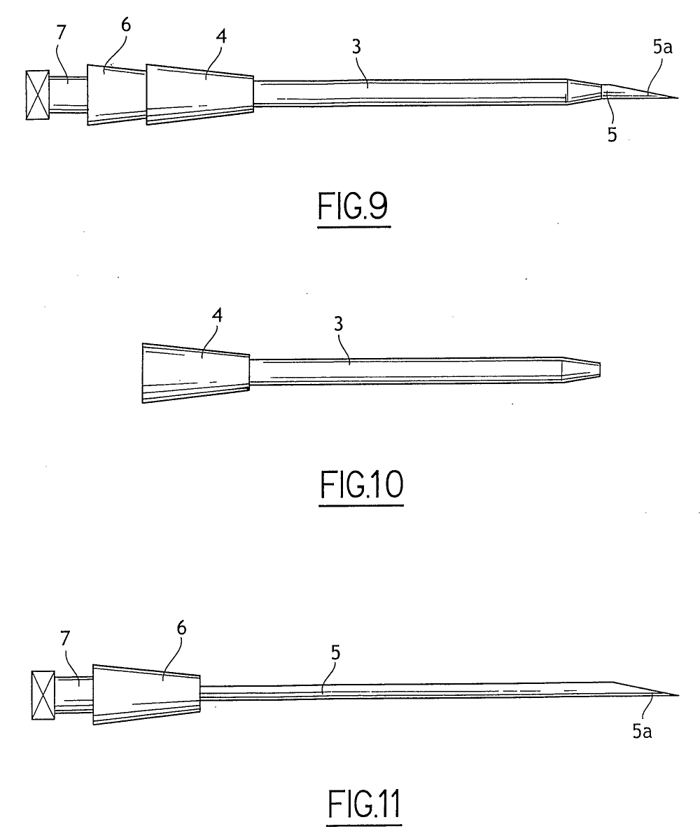

FIG. 9 is a longitudinal diagram of a known short catheter attached to a needle, before use;

FIG. 10 is a longitudinal diagram of the catheter of FIG. 9;

FIG. 11 is a longitudinal diagram of the needle of the catheter of FIG. 9;

FIG. 12 is a longitudinal diagram of a short catheter according to the invention, before use, and

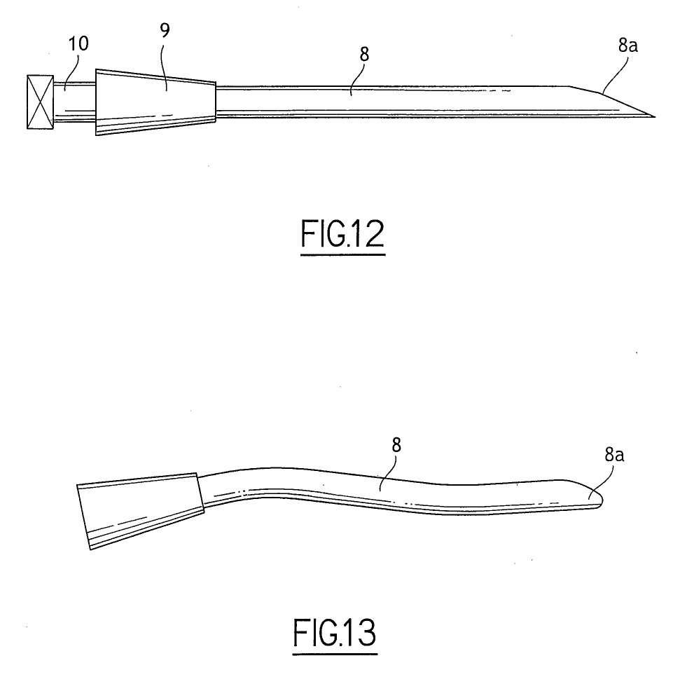

FIG. 13 is a longitudinal diagram of the short catheter of FIG. 12 after deformation.

A needle used in venous and arterial puncture generally has a cutting bevel with two types of shape;
type 1: The Lancet Point. This is a single bevel created by milling to an angle. In FIG. 1, an angle of 17° is used, though this can differ in accordance with custom and use.
type 2: The Back Cut (FIG. 3). This is a lancet-point bevel with two ground edges just below the point. This creates a narrowing of the point at the same time as a small displacement of the latter toward the centre of the tube.

On passing through the skin, a lancet-point needle creates a trace in the shape of a crescent moon. The back-cut creates a hole that is more favourable to the later passage of a cannula (e.g. with short catheter).

The deformations that are possible in the shape of the bevel in order to render it atraumatic after insertion in the vein, can include:
a dulling of all the edges of the cutting bevel by rounding,
a shortening of the length of the cutting bevel,
a thickening of the edges of the cutting bevel in addition to the rounding;
a rounding of the point of the cutting bevel,
a balling of the point of the cutting bevel of the end of the needle with the edges of the bevel returning toward the centre;
a partial flattening of the point of the cutting bevel
a curvature toward the centre of the longest side of the cutting bevel (Tuohy type needle).

The deformation is rendered possible by the appropriate choice of a shape-memory material to constitute the beveled needle-tip.

As was explained by and in the publications mentioned earlier, the use of shape memory to obtain an advantageous and controlled deformation of a tube has been known to those skilled in the art for many years, including the method for triggering this deformation and knowledge of the wanted deformation, consequently allowing the choice of the material and/or the composite structure of the tube to be deformed.

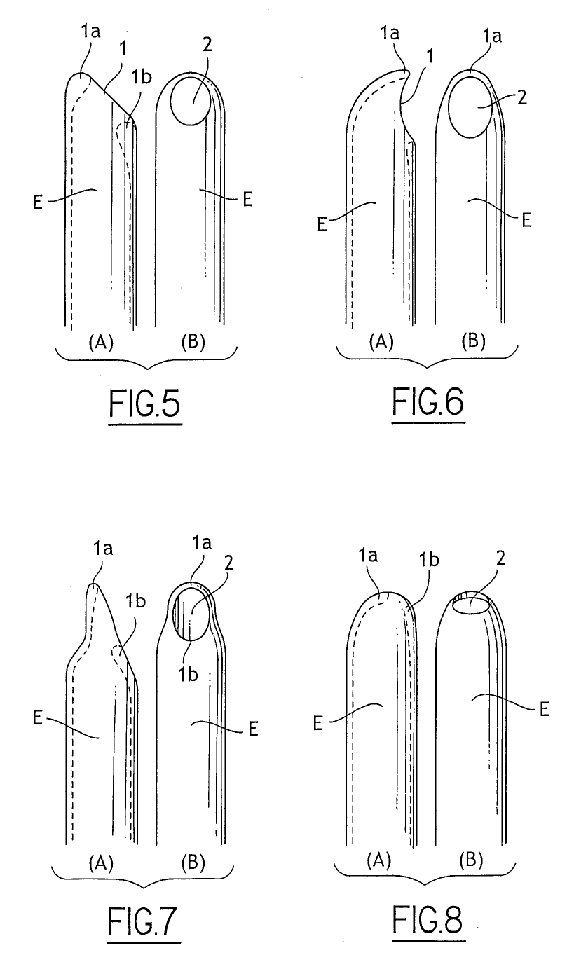

By way of an example, FIG. 2 and FIGS. 4 to 8 show deformations that are suitable for rendering atraumatic a bevelled cutting needle-tip:
FIGS. 2 and 4: the wall of the bevel (1) of the needle-tip E is rounded over the whole circumference of the orifice (2) of the bevel, and the length of the bevel is reduced. This can be accompanied by a thickening of the wall at the end;
FIG. 5: the bevel of the needle-tip E sags toward the interior of the tube;
FIG. 6: the top end (1a) of the bevel of the needle-tip E curves toward the interior and the orifice (2) is rounded;
   FIG. 7: the bevel sags toward the interior of the tube and the orifice (2) of the tube is rounded;
FIG. 8: the top (1a) and bottom (1b) ends of the bevel (1) of the needle-tip E are closer to each other.

The invention applies in particular to needles used in venous or arterial catheters or in plexus or peridural blocks and on all short catheters. In the case of the short catheter, it can be a needle+a cannula as currently, but in a preferred implementation the needle becomes an atraumatic cannula by softening.

A short catheter is usually compose of a stainless-steel tube which, at its distal end, has a cutting bevel (most often bevelled to any angle of 17°), and whose proximal end is fitted with an overmolded base equipped with an air-vent plug allowing air to pass but not liquids, and so not the blood (a water-repelling porous membrane). It is this filter which allows one, by looking at the blood reflux in the transparent air-vent plug, to check that the bevel of the tube is indeed in the vein, and that it is permissible to push the cannula. A cannula is mounted on this needle, which has to be adjusted in diameter, conical at the end (to reduce the effort of penetration into the skin) and adjusted linearly (generally the end of the cannula must be at least one millimetre from the bottom of the bevel of the metal tube).

This cannula is generally composed of a Teflon (PTFE) tube, or now most often made from polyurethane (PUR) which is heat-sensitive (it becomes flexible in the vein and can be tolerated for longer in the latter), fitted at one end with a base equipped with a lockable female syringe cone. The cannula is push-fitted onto the needle and the cannula/needle assembly can be silicone-treated (to improve slippage).

The user inserts the needle-tip, waits for the blood reflux, pushes the cannula into the vein and withdraws the needle, then connects the base of the cannula to an extender or to a perfusion line and tapes the latter to the skin (with a plaster, etc.).

As an example, FIGS. 9 to 11 show a short catheter (3) in synthetic resin, with its proximal base (4), into which a metal needle (5) with bevelled point (5a) and proximal base (6) is inserted. In this example, the needle includes, upstream of its base, a chamber for viewing the blood reflux, with its air-vent plug (7).

According to the invention, such a device can be substantially simplified if the needle and the catheter can be combined. The catheter is then composed only of a cannula, equipped with a tube, a base, and an air-vent plug mounted directly on this base. The cannula tube is in a plastic material with a very rigid shape memory and with a 17° cutting bevel (or one at any other angle) at its distal end. This tube can be silicone-treated.

After skin penetration and entry of the bevel into the vein, the trigger (the blood for example) modifies the shape of the bevel, which becomes atraumatic (non-cutting, including in the vein), the cannula is pushed in, and the tube softens as it is inserted.

The air-vent plug is removed, the extender or the perfusion line is connected, and the base is secured (with a plaster or other means).

It is then possible to remove a cannula that is flexible and atraumatic.

With such a short catheter, one saves on the needle, which is very important in terms of cost (the cost of the needle itself and costs of adjusting the cannula on the needle), the number of operations is reduced (no needle to remove), as are the risks of accidental pricking (nosocomial infections).

As an example, FIG. 12 shows a tube in synthetic resin with shape memory (8) with its base (9) and a viewing chamber (10) fixed in a detachable manner on the base of the tube. The tube is bevelled at its distal end (8a) (this bevel can be preceded by a gentle slope) and is made from a shape-memory material so that, before deformation, the tube is sufficiently rigid to act as a needle with a view to puncture, and the insertion of its point into a vein, and then, after insertion, the tube develops a blunted end and a softened and flexible tubular part like a conventional catheter.

The invention can be applied to simple needles whose tube can be made from a rigid plastic material with a bevel, where this material also has shape memory. The needle is inserted and the cutting end in the skin or the vein or the artery becomes atraumatic (the trigger is the liquid), and it is even possible to use the needle as a cannula in the vein or the artery (for more reliable positioning).

When the needle is withdrawn, there is then no risk of accidental pricking.

To this end, a very rigid plastic material is required, which can be cutting, with a moulded bevel shape.

This material has to be very fluid at the moment of injection-moulding in order to fill all shapes in the very fine moulds.

There already exist plastic materials that are very rigid (Polyethyletherketone (PEEK), polyimide, etc.), and one can imagine that it will be possible to further increase this rigidity. The plastic material can even be suitable for milling or turning (like metal).

### EXAMPLE

The shape-memory material (M) is composed of two materials:
M1 which softens at high temperature and is rather soft;
M2 which softens at low temperature and is very hard.

Tube M is pushed into in a high-temperature mould in order to impart to it the chosen atraumatic shape. On cooling, this shape is imposed at M1 and at M2.

The tube is pushed into a second mould at low temperature, sufficient to soften M2 but not M1. It is then also possible to insert a rod into the tube in order to reform the internal shapes of the first moulding.

On cooling the very hard M2 imposes the shape of M1, the second moulding.

It is also possible to mill the shape in order to render it sharper.

When the puncture assembly is pushed into the vein, either the temperature of 37° C. is sufficient to soften M1, but is somewhat low (to allow for the preliminary sterilization temperature (50° C.), the milling process which can heat it, storage in summer, etc.), or the hard material is very hydrophilic and softens very quickly in contact with saline water (like contact lenses), thus allowing M1 to impose its shape, or a hot or electrically-heatable mandrel can be inserted through the whole puncture device in order to soften M2.

The invention is not limited to the implementation examples that have been described.

1. A tip that is usable in the medical field to pierce the skin and penetrate into a site of the body, said needle-tip being composed of a section of hollow tube in a shape-memory material that, at its distal end, has a stable and rigid cutting bevel, designed to cut the skin and penetrate into the site, wherein the cutting bevel remembers a state in which it is not sharp, and in that it is capable of changing state so as to return to an atraumatic state when it is subjected to a condition that is prevalent at the site, or under the action of a stimulus controlled externally.

2. The needle-tip according to claim 1, whose material is chosen so that the change of state of the bevel is triggered by a rise in temperature within the site.

3. The needle-tip according to claim 1, whose material is chosen so that the change of state is triggered by contact with a liquid within the site.

4. The needle-tip according to claim 1, whose material is chosen so that the change of state is triggered by an electrical stimulus controlled from the exterior of the site.

5. The needle-tip according to one of claims 1 to 4, whose bevel when not sharp has a shape which differs from the shape of the cutting bevel by one or more of the following changes—dulling of all the edges of the cutting bevel, shortening of the length of the cutting bevel, thickening of the edges of the cutting bevel, rounding of the point of the cutting bevel, balling of the point of the cutting bevel, partial flattening of the point of the cutting bevel, or curvature of the longest side of the cutting bevel toward the centre.

6. The needle-tip according to claim 1, composed of materials designed to be heated to allow moulding of the needle-tip to the desired atraumatic shape, and designed to allow the bevel of the needle-tip to be stressed so that it adopts the cutting shape, where one of these materials is designed to keep the bevel of the cooled needle-tip in the cutting shape for as long the needle-tip is not subjected to the said condition or to the said stimulus.

7. The needle-tip according to claim 1 created from polymer materials.

8. The needle-tip according to claim 1 whose cutting bevel is of the “Lancet Point” type.

9. The needle-tip according to claim 1 whose cutting bevel is of the “Back-Cut” type.

10. The needle-tip according to claim 1, in which the cutting bevel is preceded by a gentle slope.

11. The needle-tip according to claim 1, which constitutes one end of a tube.

12. A tube fitted with a needle-tip according to claim 1, manufactured together with the tube in a shape-memory material so that it can pass, under the effect of a condition that exists at a site, from a state in which the tube has the rigidity required so that the needle-tip can be pushed as far as the site, to a state in which the tube is flexible.

13. The needle-tip according to claim 5, composed of materials designed to be heated to allow moulding of the needle-tip to the desired atraumatic shape, and designed to allow the bevel of the needle-tip to be stressed so that it adopts the cutting shape, where one of these materials is designed to keep the bevel of the cooled needle-tip in the cutting shape for as long the needle-tip is not subjected to the said condition or to the said stimulus.

14. The needle-tip according to claim 5 created from polymer materials.

15. The needle-tip according to claim 5 whose cutting bevel is of the “Lancet Point” type.

16. The needle-tip according to claim 5 whose cutting bevel is of the “Back-Cut” type.

17. The needle-tip according to claim 5, in which the cutting bevel is preceded by a gentle slope.

18. The needle-tip according to claim 5, which constitutes one end of a tube.
